(19) United States
(12) Patent Application Publication
(10) Pub. No.: US 2023/0243721 A1
(43) Pub. Date: Aug. 3, 2023

(54) RESIDUE COLLECTION

(71) Applicant: Verifeque Innovations, Inc., Wilmington, DE (US)

(72) Inventor: Christian Loane, Carson City, NV (US)

(21) Appl. No.: 18/002,838

(22) PCT Filed: Jun. 23, 2021
(86) PCT No.: PCT/US2021/038685

§ 371 Date: Dec. 21, 2022

Related U.S. Application Data
(60) Provisional application No. 63/042,768, filed on Jun. 23, 2020.

(51) Int. Cl. G01N 1/02; G01N 33/52

(52) U.S. Cl. CPC G01N 1/02; G01N 33/528; G01N 2001/028; G01N 2001/4061

(57) ABSTRACT

A sample residue collection device is provided. The sample residue collection device can include a housing, a nib, and a liquid or solvent configured for receipt within the nib. The housing can include a body, a nib housing, and an enclosure cap. The device can be configured for detection and identification of samples residues by colorimetric indications. The device can optionally include a solvent reservoir positioned within the body and containing at least a portion of the liquid or solvent. The solvent reservoir can be in fluid communication with the nib. The housing and nib with solvent may be single use or interchangeable and replaceable, providing a multiple use device. Related apparatus, systems, techniques, and articles are also described.

### CROSS-REFERENCE TO RELATED APPLICATIONS

This application claims the benefit of U.S. Provisional Patent Application No. 63/042,768, filed on Jun. 23, 2020, and entitled “Residue Collection,” the entirety of which is incorporated by reference.

### BACKGROUND

A pen is a common writing instrument used to apply ink to a surface, usually paper, for writing or drawing. Historically, reed pens, quill pens, and dip pens were used, with a nib dipped in ink. Modern types of pens typically can include a reservoir of ink feeding a point, such as a ballpoint, rollerball, fountain, and felt or ceramic tip.

A swab (e.g., cotton swab, or cotton bud) is a handheld item that includes one or two small wads of cotton wrapped around one or both ends of a short rod made of a wood, rolled paper, plastic, and the like. Uses include first aid, cosmetics application, cleaning, and arts and crafts. These devices may be similarly designed for surface residue collection.

### SUMMARY

U.S. Pat. No. 6,677,291 describes a pen like device for the dispensing of solvents to a surface for cleaning applications including glue from electronic circuitry. The device as described is not for narcotic, explosive and general chemical agent detection, and does not deliver solvent from the housing for safe solid residue collection.

U.S. Pat. No. 5,702,759 describes a pen like device for dispensing hazardous acidic liquids to a surface for the repair of damaged surfaces. Specifically for the repair of aluminum surfaces. The device as described is not for narcotic, explosive and general chemical agent detection, and does not deliver solvent from the housing for safe solid residue collection.

CN 101393135 describes a pen-like structure with pre-wetted paper filled core, which detects the pH of surfaces or ambient atmosphere, which the nib comes into contact with. The device as described is not for narcotic, explosive and general chemical agent detection, and does not deliver solvent from the housing for safe solid residue collection.

U.S. Pat. No. 5,960,802 describes a pen like device for the dispensing of chemicals to a surface including the application of growth hormone solution to the scalp to enhance hair growth. The device as described is not for narcotic, explosive and general chemical agent detection, and does not deliver solvent from the housing for safe solid residue collection.

JP 4798212 describes a pen like device for electronic surface writing, timing, and recording. The device as described is not for narcotic, explosive and general chemical agent detection, and does not deliver solvent from the housing for safe solid residue collection.

US Patent Publication 2019/0168210A1 describes a screw top vial like device for receiving a volume of human blood directly from the patient. The device as described is not for narcotic, explosive and general chemical agent detection, and does not deliver solvent from the housing for safe solid residue collection.

U.S. Pat. No. 3,457,014 describes a pen like device for writing. Contained within the housing core is a solid residue, which is subsequently dissolved when a volatile liquid enters the core, and then squeezing the sides of the core, the liquid ink is dispensed from a nib. The device as described is for surface ink marking purposes and not for sample residue collection.

U.S. Pat. No. 6,548,018 describes a syringe type device, containing liquid colorimetric reagents, which are expelled from the end of the device onto a surface facilitating a colorimetric indication on the surface. The device as described is not safe sample residue collection.

U.S. Pat. No. 6,991,898 describes a pen like device for which contains liquid colorimetric reagents within the housing core. The device is designed for inserting into the body cavity and performing colorimetric detection of microorganisms, viewing color indications through clear panels on the side of the pen housing. The device as described is not for narcotic, explosive and general chemical agent detection, and does not deliver solvent from the housing for safe solid residue collection.

U.S. Pat. No. 7,297,729 describes a pen like device with and ink and film forming composition for marking a surface. The device as described is for surface ink marking purposes and not for sample residue collection.

U.S. Pat. No. 8,317,728 describes a swab for collecting biological specimens and a vial for inserting the swab for culture development. The device as described is not for narcotic, explosive and general chemical agent detection, and does not deliver solvent from the housing for safe solid residue collection.

U.S. Pat. No. 8,216,832 describes a single use swab for biological pathogen analysis, with an extendable telescopic swab tip and protective layers over the swab tip. The device as described is not for narcotic, explosive and general chemical agent detection, and does not deliver solvent from the housing for safe solid residue collection.

U.S. Pat. No. 8,745,806 describes a pen style device for dispensing liquids from an ejectable nib for cleaning surfaces. The device as described is not for sample collection.

US Patent Publication 2004/0220748A1 describes a swab like device for surface collection of biological samples and chemical reagents within the housing core for colorimetric indication of said biological agents. The device as described is a conventional swab, and performs the colorimetric reaction within the swab structure as an all in one device, and is not suitable for multi-use purposes.

US Patent Publication 2013/0118275A1 describes a swab style device with releasable sponge nib for surface sample collection. The device as described fails to deliver solvent from pen for solid residue collection.

International Patent Publication WO 2012/145379A1 describes a pen like device with dry porous nib for liquid sample collection. The device as described fails to deliver solvent from pen for solid residue collection.

Examples of commercially available refillable ink and replaceable nib marker pens are described in the following links: Risk Reactor's MRA-X (available from Risk Reactor, Inc.® of Santa Ana, Calif.), Blick Arts Holbein® refillable marker pens (available from Dick Blick Holdings, Inc. of Galesburg, Ill.), and Copic® replacement nib marker pen housings (available from Too Marker Products Inc. of Tokyo, Japan) are example of commercially available pens for marking and writing applications. The MRA-X and Holbein® markers are hollow barrel construction, made of high-grade aluminum alloy and Polypropylene plastic respectively. Both have a valve O-ring seal system to prevent ink leakage and a threaded cap on the barrel, which is removed during ink re-filling. Copic® markers contain a non-removable ink capillary. All three marker examples allow replacement of felt nibs, available in Round, Chisel, Fine Line Round and Fine Line Chisel shapes. The MRA-X and Holbein® markers are refilled by unscrewing the end cap from aluminum handle and pouring ink into the aluminum handle and replacing the cap firmly back on to aluminum handle. All markers require the nibs to be removed and replaced by hand, pulling out old nibs and pushing in new nibs at the marker tip. All markers are used by holding in the writing position and with a quick motion, press down on the tip, allowing ink to flow, release and write.

In an embodiment, a sample residue collection device is provided. The sample residue collection device can include a housing, a nib, and a liquid or solvent configured for receipt within the nib. The housing can include a body, a nib housing, and an enclosure cap. The device can be configured for detection and identification of samples residues by colorimetric indications.

One or more of the following features can be included in any feasible combination.

In another embodiment, the device can further include a solvent reservoir positioned within the body and containing at least a portion of the liquid or solvent, wherein the solvent reservoir is in fluid communication with the nib.

In another embodiment, the device can be in the form of a pen. The pen can be composed of plastic and metal and combinations thereof. The body can be hollow and able to store the solvent reservoir in the form of a capillary or bibulous carrier. The solvent reservoir can include a capillary composed of natural and/or synthetic fibers. The capillary can include at least one of wool, cotton, cellulosics, synthetic fibers, rayon, nylon or esters. The liquid or solvent utilized can include at least one of water, dimethyl sulfoxide, aliphatic and aromatic alcohols, essential oils or terpenes. The solvents can include an essential oil. The essential oil can include a Eucalyptus sp. Oil. The Eucalyptus sp. Oil can include at least one of Globulus, Blue Mallee, Lemon, or Radiata. The solvents can include a melaleucal oil. The melaleucal oil can include a Melaleuca Alternifolia oil.

In another embodiment, the nib housing can connect into the body, thereby supporting and holding the capillary in place. The nib can be constructed of a porous material including synthetic or natural fibers that allow saturation and wicking of solvents and encapsulation of sample residue. The nib can be constructed from one or more of wool, cotton, cellulosics, synthetic fibers, rayon, nylon, esters, thermally bonded fibers, porous ceramics or metals.

In another embodiment, a tip of the nib can formed in a shape of a bullet, slanted chisel, flat chisel, cone, or step down bullet. The nib can be inserted into and supported by the nib housing, protruding in such a way as to allow dabbing and rubbing enabling residue sample collection. The nib can be removably coupled to the nib housing.

In another embodiment, the cap can be configured to couple to the body, thereby covering the nib. The cap can be configured to protect the nib during post manufacture packaging, shipping, storage and final use. The cap can reduce evaporation of the solvent. The body and nib housing, as well as the body and the cap can be fitted and held together by at least a male-female thread, pressure joining, or clip seal mechanisms.

In another embodiment, the safety of end users can be greatly enhanced when sampling dangerous and hazardous substances as the need to scoop, wipe and perform multi-step sample collection methods by hand is reduced to a single step. The nib can protrude from the nib housing, extending inwards through the nib housing into the body contacting the solvent reservoir (e.g., capillary) which allows flow of solvent from the solvent reservoir (e.g., capillary) into the nib and on contact of the wetted nib with a surface sample residue, wicking and dispersive forces allow the solvent to wet residue particulate preventing airborne particulate formation. End user safety can be enhanced due to the prevention of airborne particulate landing on end user skin surfaces during sampling.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The benefits and advantages of the present invention will become more readily apparent to those of ordinary skill in the relevant art after reviewing the following detailed description and accompanying drawings, wherein:

FIG. 1 is a schematic diagram illustrating an exploded view of an exemplary embodiment of an industrial style residue collection device including a housing and a nib;

FIG. 2 is a schematic diagram illustrating an exemplary embodiment of a “high end/fashion style” residue collection device including a housing and a nib;

FIG. 3 is a schematic diagram illustrating an exemplary embodiment of a “marker style” residue collection device including a housing and a nib;

FIG. 4A. is a schematic diagram illustrating another exemplary embodiment of a residue collection device including a housing having a body, a nib housing, and a cap;

FIG. 4B is a schematic diagram illustrating the residue collection device of FIG. 4A including a nib;

FIG. 4C is a schematic diagram illustrating a side cross-sectional view of a first half of the residue collection device of FIG. 4A; and

FIG. 4D is a schematic diagram illustrating a side cross-sectional view of a second half of the residue collection device of FIG. 4A.

Like reference symbols in the various drawings indicate like elements.

It is noted that the drawings are not necessarily to scale. The drawings are intended to depict only typical aspects of the subject matter disclosed herein, and therefore should not be considered as limiting the scope of the disclosure. Those skilled in the art will understand that the systems, devices, and methods specifically described herein and illustrated in the accompanying drawings are non-limiting exemplary embodiments and that the scope of the present invention is defined solely by the claims.

### DETAILED DESCRIPTION

There is a need for a residue collection device housing and nib combination that can be safely used for sample collection and in combination with colorimetric reagents, facilitate the detection and identification of unknown compounds. Accordingly, embodiments of the present disclosure relate to collection of residue, and related apparatus, systems, techniques, and articles. As discussed in detail below, the disclosed embodiments can be employed for use in the collection of sample residues and mixing of said residues with colorimetric reagents to facilitate a colorimetric reaction for the detection and identification of unknown substances.

As described above, a typical pen includes a reservoir of ink that feeds a tip, enabling placement of the ink onto a surface in a controlled manner (e.g., for writing). A swab can include a fixed, non-removable cotton or other fiber matrix tip and one or more chemical reagents within swab construction that, while it can be used to obtain a sample of a substance from a surface, is at best single use and produces colorimetric reactions both on surfaces where residues are found or within the swab tip or within the swab housing.

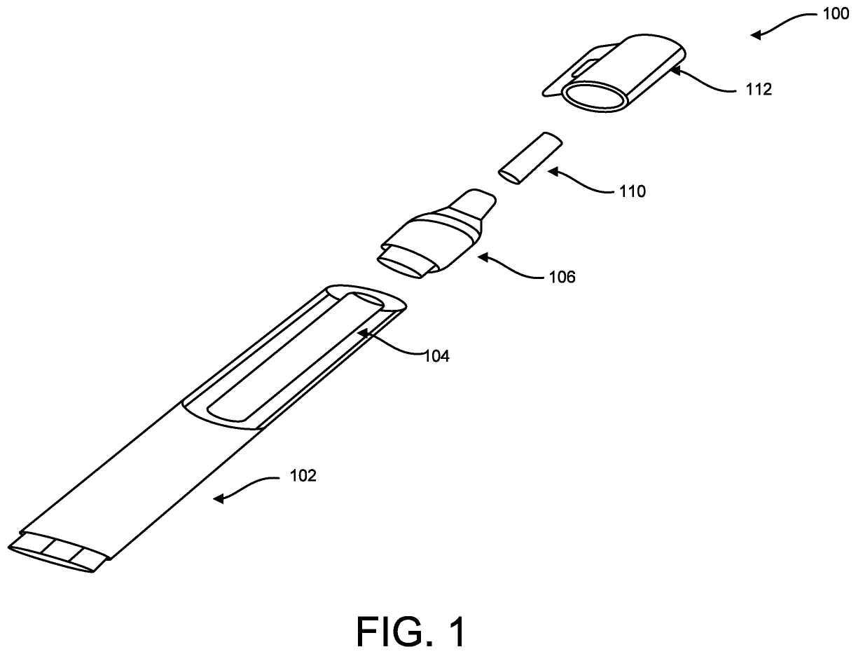

Some implementations of the current subject matter include a portable device for sample collection. For example, a portable hand held device can take the form of a residue collection device 100, as illustrated in FIG. 1, and can include a housing including a body 102, a nib housing 106, and a cap 112. These components of the housing can be independently formed from plastics (e.g., polypropylene, polycarbonate, polyvinyl chloride, fluorinated polymers, etc.), metals (e.g., aluminum), ceramics, and combinations thereof.

The housing can further contain a nib 110 which dispenses a liquid, enabling safe wetting, dissolution and collection of sample residues for analysis. In particular the residue collection device 100 and/or nib 110 can be filled with a liquid capable of safely wetting, dissolving, collecting and transferring a wide variety of solid, liquid, aerosol, gel and cream residues, including but not limited to chemical and biological agents, narcotics, explosives, environmental contaminants and their pre-cursors. The residue collection device 100 and nib 110 can be compatible with a wide range of liquids, which may be aggressive, hazardous and/or volatile and so the residue collection device 100 construction can prevent the liquids from spoiling and/or evaporating prior to use.

Exemplary liquid(s), which may be utilized for sample residue and colorimetric reagent wetting, dissolution, collection and mixing can include, but are not limited to, water, dimethyl sulfoxide, aliphatic and aromatic alcohols, essential oils and terpenes.

Without limitation, embodiments of the nib 110 can be formed from materials including, but are not limited to, wool, cotton, cellulosics, synthetic fibers, rayon, nylon, esters, polyester, thermally bonded fibers, porous ceramics, metals, and combinations thereof. The nib tips may be presented in a variety of shapes, including but not limited to, bullet, slanted chisel, flat chisel, cone, and step down bullet. Other form factors are possible.

In certain embodiments, the liquid can be stored within the body 102 (e.g., within a capillary reservoir 104 in fluid communication with the nib 110). Without limitation, embodiments of the capillary reservoir 104 can be formed from materials including, but not limited to, wool, cotton, cellulosics, synthetic fibers, rayon, nylon, esters and any combination thereof. In alternative embodiments, the capillary reservoir can be omitted and the nib can be provided in a pre-wetted state containing the liquid.

By way of example and without limitation, a liquid can be an essential oil, and in particular the essential oil is Eucalyptus sp. oil or Tea Tree oil. An essential oil can be provided within a dispenser for sample residue collection.

In an embodiment, the portable device can be in the form of a pen, as described within. By way of example and without limitation, the nib 110 can include a bullet-shaped nib tip, composed of soft synthetic fiber (e.g., polyester). In certain embodiments, the nib can be provided as one integral component of a complete dispenser and the dispenser can be in the form of a pen, which can be designed for the collection of sample residues and mixing of said residues with colorimetric reagents to facilitate a colorimetric reaction for the detection and identification of unknown substances.

A present liquid solvent, capable of sample collection by way of wetting and/or dissolution of said sample and subsequently wetting colorimetric regents, allowing mixing and there by facilitating a colorimetric reaction includes essential oil(s). The essential oil(s) is present as Eucalyptus sp. oil. Many Eucalyptus sp. oils are known, including but not limited to Globulus, Blue Mallee, Lemon, Radiata. An exemplary oil is the Eucalyptus sp. Lemon oil.

In yet another example of the present liquid as an essential oil(s) is Tea Tree oil, from the Myrtaceae family. An exemplary genus of Myrtaceae is the Melaleuca, and in particular Melaleuca Alternifolia oil.

Referring now to the figures, and in particular to FIG. 1, there is shown a residue sample collection device 100, also referred to herein as residue collection device 100. The residue sample collection device 100 can be configured for sample collection, transfer and mixing of samples with colorimetric reagents to facilitate a colorimetric indication for identification of unknowns. Embodiments of the sample collection device are illustrated in FIGS. 1-3 as a residue collection device, which will be discussed in more detail below.

An essential oil in accordance with the disclosed embodiments has been found to be useful for a wide range of applications. For example, it has been found that embodiments of the essential oils can be useful in drugs of abuse sample collection, for example, penetrating and wetting powdered Fentanyl Analogue (FA) residue, thus preventing powdered particulate becoming airborne during sampling and subsequent analysis. It has also been found that the embodiments of the essential oils can solubilize Fentanyl Analogues, allowing wicking and uptake of the FA powders, thus removing the need for frontline personnel coming into contact with these extremely dangerous substances. It has also been found that embodiments of the essential oils can readily solubilize colorimetric reagents, which are provided as dry powders, or liquids, or printed onto a solid substrate, allowing mixing of said colorimetric reagents with the solubilized FA, thus facilitating a colorimetric indication, allowing detection and identification of FA.

The essential oil (e.g., Eucalyptus oil and the like), can exhibit less aggressive solvent characteristics, as well as less offensive odors, than many traditional solvents. These solvents are readily available and have good compatibility with a wide range of storage and/or delivery housing compositions, including but not limited to metal, aluminum, plastics e.g., polypropylene, polycarbonate, polyvinyl chloride, and fluorinated polymers.

All of the above characteristics can be desirable in a simple, portable, mass produced sample collection device.

Returning again to FIG. 1, the residue collection device 100 can be configured for use with embodiments of the liquid essential oil (EO). The residue collection device 100 includes the body 102 defining a sealed solid or hollow chamber which may or may not contain the capillary reservoir configured to store the EO. In certain embodiments, the capillary reservoir can be pre-wetted. The hollow nib-housing 106 can be mounted to the body 102 (e.g., by male-female thread or clip-lock mechanism). The nib 110 can be formed from a porous material, such as felt or other materials as discussed above. A portion of the nib 110 can be further inserted into the nib-housing 106, abutting the capillary reservoir 104, placing the nib in fluid communication with the capillary reservoir and allowing wicking and wetting of the nib 110 with the EO contained in the capillary reservoir 104.

The cap 112 can be configured to affix to the body 102, thereby covering the nib 110 and the nib housing 106. So configured, the cap 112 can protect the nib 110 during post production packaging, shipping, use and can prevent excessive evaporation of embodiments of the EO, enhancing shelf life of product. In certain embodiments, the cap can be secured to body by a clip lock mechanism. Other mechanisms are also envisioned. A portion of the nib 110 can protrude from the nib housing 106, extending inwards through the nib housing 106 into the body 102 chamber to contact the capillary reservoir 104, which allows flow of the EO from the capillary reservoir 104 into the nib 110, by wicking or capillary action. Contact of the marking end of the nib 110 (e.g., opposite the end of the nib contacting the capillary reservoir 104) with a surface residue allows the EO to flow by capillary and dispersive forces into the surface residue particles and or molecular matrix, thereby allowing dissolution and solubilization mechanisms to lift the sample solute and wick into a porous matrix of the nib 110, achieving sample collection.

In certain embodiments, the residue collection device may be configured for single use. In this format, the capillary reservoir 104 may be of reduced size or not present at all. The nib 110 may be permanently fixed to nib housing 106 and the body 102 and the nib housing 106 may be formed as individual components or as a single piece, blow or injection mold plastic unit.

In alternative embodiments, the residue collection device may be configured for multiple use. In this format, the capillary reservoir 104 can be of a size which ensures adequate wicking of the essential oil for repeated sample collection with numerous fresh replaceable nibs D. The nib 110 can be configured to be firmly fixed to nib housing 106, but further configured to allow ease in removal of a used nib, and insertion of a new nib. The body 102 and the nib housing 106 may be formed as individual components or as a single piece, blow or injection mold plastic unit.

Embodiments of the nib 110 can have a sampling end or tip designed in bullet, chisel, wedge, or fine point shapes to enhance sample residue collection and mixing with colorimetric reagents.

As noted above, permanent or temporary ink marking of a surface and or cleaning dirt and/or glue from surfaces can be achieved by pens, for example, ITW Mark-Tex® of Roseland, N.J., Micro Care® Corporation of Bristol, Conn. under the trademark Tidy Pen®, and similar markers and pens sold under the Newell Brands®. But these pens do not allow and are not intended for sample collection of residue for detection and identification of unknown substances.

Some embodiments of the portable residue collection device 100 can be configured to allow safe collection and transfer of solid, liquid, aerosol, gel and cream sample residue. The sample residue can be contained within the nib 110 and then transferred, to a paper strip, or similar, containing a colorimetric reagent(s), which react, when mixed together, with the sample residue, thereby developing a colored end reaction product, which may identify the class of chemicals present in the sample residue.

The portable residue collection device 100 (for example, the housing and/or the nib 110) may be constructed for single use or multiple use by simply removing the used nib and exchanging it for a new nib.

The portable residue collection device 100 (for example, the housing and/or the nib 110) may be filled with any liquid which facilitates safe collection, dissolution and transfer of said sample residue.

The portable residue collection device housing may be constructed in any configuration which enables liquid storage within its structure and also allows the nib 110 to be removed and exchanged. In some implementations, the housing can include the cap 112 to cover and protect the nib 110, enhancing safe packaging, shipping and storage and prevent solvent evaporation and contamination of the nib 110 prior to use.

The nib 110 can be constructed in such a way as to allow wicking and dispensing of liquid(s). It can be soft, yet rigid to allow surface rubbing, for collection of residues and mixing of chemicals. In some implementations, the nib can be formed from a porous material having a matrix that can include a bibulous fiber matrix composed of either natural or synthetic fibers, which can be compatible with a wide variety of liquids and chemicals.

In particular the residue collection device 100 and/or the nib 110 can be filled with a liquid capable of safely wetting, dissolving, collecting and transferring a wide variety of solid, liquid, aerosol, gel and cream residues, including but not limited to chemical and biological agents, narcotics, explosives, environmental contaminants and their pre-cursors. In some embodiments, the residue collection device 100 and/or nib 110 can be compatible with a wide range of liquids which may be aggressive, hazardous and/or volatile and so the construction of the residue collection device 100 can prevent the liquids from spoiling and/or evaporating prior to use.

As noted above, commercially available refillable ink and replaceable nib marker pens are available and include but are not limited to Risk Reactor, Dick Blick, and Copic®. These pens are for writing and marking purposes only, and do not allow and are not intended for sample residue collection.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

In some embodiments, the residue collection device can be included in a kit with one or more substance containing compositions for colorimetric indication of substances. For example, multiple use sample collection device as described above may be packaged with different replaceable soft, bullet shaped, felt nibs (e.g., three nibs). Each nib can be designed to facilitate the detection of differing type of narcotic class. In one example, the nib can be pre-wetted with Tea Tree Oil. This nib can be placed into the pen-shaped residue collection device housing, and held like a marker pen. The nib can be rubbed through suspect residue on a surface. This collected sample can now be transferred to a colorimetric detection strip, for example a Swabtek® Fentanyl Test card (available from www.swabtek.com, Veriteque USA, Inc., of Carson City, Nev.). The nib can be rubbed and dabbed onto the test card test zone for detection and identification of Fentanyl Analogue residues.

In another embodiment, the used nib from the above test can be removed and disposed; a new second nib containing Eucalyptus Oil can be placed into the pen shaped residue collection device housing; held like a marker pen; and the nib can be rubbed through suspect residue on a surface. This collected sample can now be transferred to a colorimetric detection strip, for example a Swabtek® Cocaine Test card (available from www.swabtek.com, Veriteque USA, Inc., of Carson City, Nev.). The nib can be rubbed and dabbed onto the test card test zone for detection and identification of cocaine residues.

In yet another embodiment, the used nib from the above test can be removed and disposed correctly, a new dry nib can be placed into the pen shaped residue collection device housing; held like a marker pen; and the nib rubbed through vaping liquid (e.g., juice or e-liquid) residues. This collected sample can now be transferred to a colorimetric detection strip, for example a Swabtek® Nicotine Plus card (available from www.swabtek.com, Veriteque USA, Inc., of Carson City, Nev.). The nib can be rubbed and dabbed onto the test card test zone for detection and identification of clandestine narcotic residues in vaping liquid formulations.

Although a few variations have been described in detail above, other modifications or additions are possible. For example, the residue collection device may be designed for single use application, and composed of biodegradable components.

In another example the residue collection device may be designed for first responder and military application in a ruggedized industrial “marker pen” style.

In yet another example, the residue collection device may be designed for home use and resemble more a traditional “ball point pen” style.

In yet another embodiment, the residue collection device may be designed for discrete use and resemble more a traditional “cosmetic eye liner” style.

The subject matter described herein provides many technical advantages. For example, in the current device as described, the device housing may be constructed for multiple use. The nibs may contain any type of solvent system for any type of suspect residue collection. In this way the device allows for the insertion and use of specific nibs for specific applications, with the ability of unlimited nib replacement. In this way, waste generation drastically minimized, when compared to that of traditional single use analytical swabs.

In another embodiment the residue collection device housing may be constructed completely of biodegradable plastics, further reducing waste streams.

In yet another embodiment, once the sample has been collected, the nib cap may be replaced, and sample safely stored within the nib for later testing on the appropriate type of test card.

In yet another embodiment, the size and style of the device housing allows it to be used by many consumer groups with varying environmental and operational conditions. For example, a parent at home, a police officer, a fire department officer and military personnel have very different operational conditions. Time, weather, fire, hands gloved or un-gloved, with and without helmets and life threatening variables may be catered for in, for example but not limited to, the overall design, housing shape and size, nib shape and size, device cap re-sealing for safe storage and transfer of collected sample.

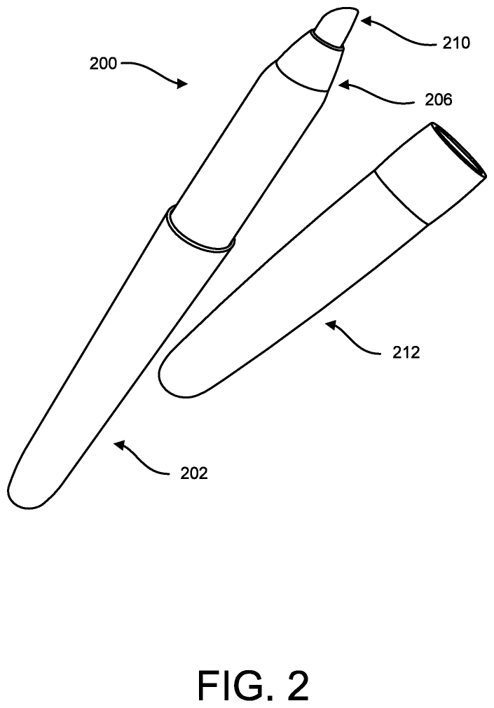

FIG. 2 illustrates another embodiment of the residue collection device in the form of residue collection device 200. The residue collection device 200 can include the housing in a “high end/fashion style” shape including body 202, nib housing 206, nib 210, and cap 212. The functionality of the body 202, nib housing 206, nib 210, and cap 212 can be as discussed above with respect to the body 102, nib housing 106, nib 110, and cap 112. Embodiments of the body can omit or include the capillary reservoir, as discussed above. The cap 212 can be a screw cap configured to secure to the body 202 by mating male-female threads.

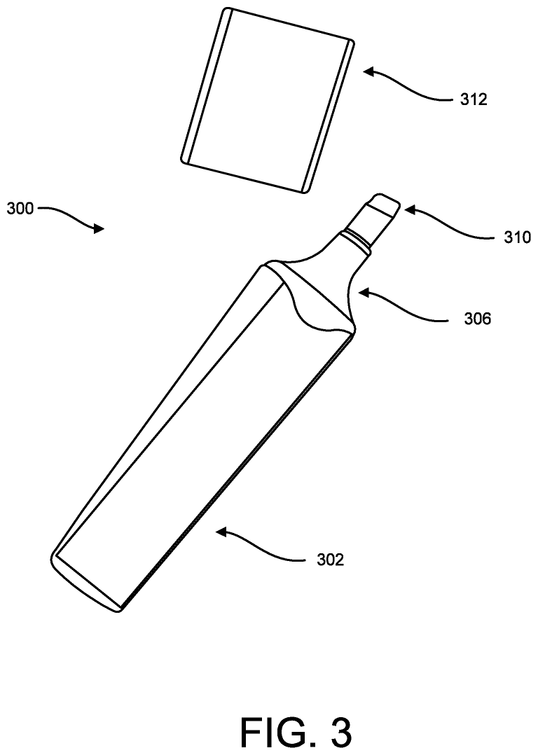

FIG. 3 illustrates another embodiment of the residue collection device in the form of residue collection device 300. The residue collection device 300 can include the housing in a “marker” shape including body 302, nib housing 306, nib 310, and cap 312. The functionality of the body 302, nib housing 306, nib 310, and cap 312 can be as discussed above with respect to the body 102, nib housing 106, nib 110, and cap 112. Embodiments of the body can omit or include the capillary reservoir, as discussed above. The cap 312 can be a configured to secure to the body 202 a clip-lock mechanism or by mating male-female threads.

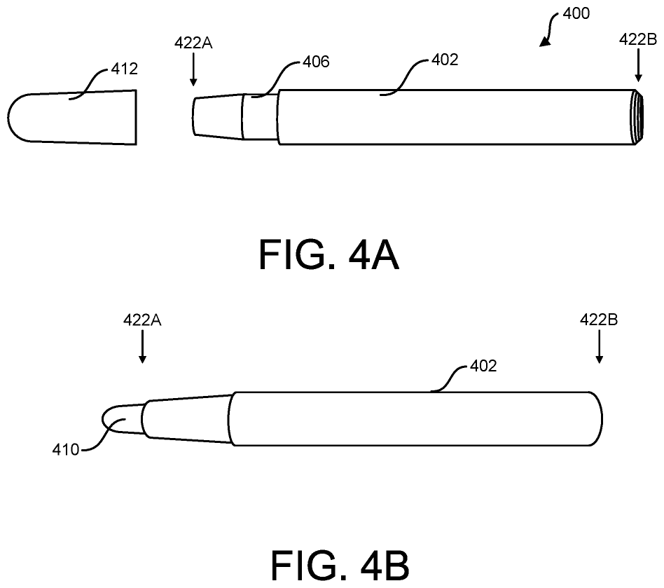

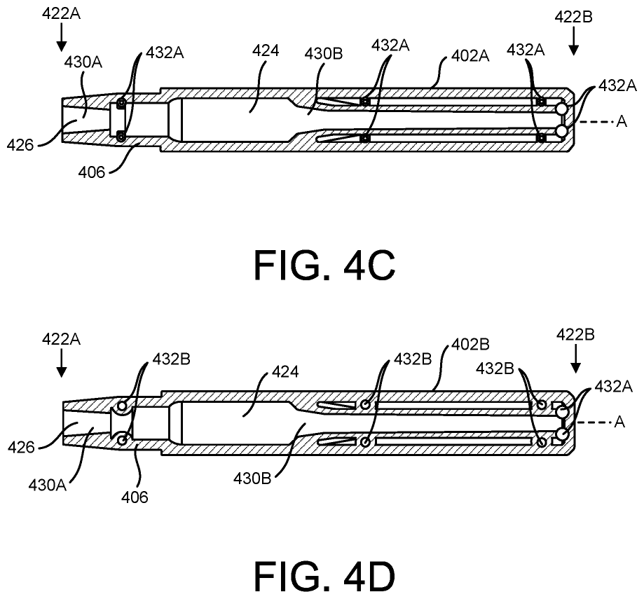

Another exemplary embodiment of a residue collection device in the form of residue collection device 400 is illustrated in FIG. 4A-4D. As shown, the residue collection device 400 is provided in a pen-shaped form factor that includes a housing having a body 402 and a cap 412. The body 402 and the cap 412 can be independently formed from materials such as those discussed above with regards to the housing of the residue collection device 100.

FIG. 4B further illustrates the residue collection device 400 with a nib 410 that is secured to the body 402 at a nib mounting portion 406. Retention of the nib 410 within the body 402 is discussed in greater detail below.

In certain embodiments, the body 402 can be formed as two halves, a first half 402A and a second half 402B, that are configured to couple together. As shown, the first half 402A includes one or more first coupling features 432A and the second half 402B includes one or more corresponding second coupling features 432B. In one example, the coupling features 432A, 432B can be posts and sockets. Such posts and sockets can provide a friction fit between the first and second halves 402A, 402B when the posts are inserted within the sockets. It can be appreciated, however, that other mechanisms can be employed to secure the first and second halves 402A, 402B together, alone or in combination with the first and second coupling features 432A, 432B. Examples can include, but are not limited to, adhesives, clips, and the like.

As further illustrated in FIGS. 4C-4D, a channel 424 can be formed within the body, extending along a longitudinal axis A between first and second ends 422A, 422B of the body 402. The channel 424 can be open at the first end 422A of the body 402 and closed at the second end 422B of the body 402. So configured, an opening 426 is defined within the first end 422A of the body 402.

In certain embodiments, the residue collection device 400 can be configured to employ a cotton swap as the nib 410. That is, a cotton swab can be inserted within the body 402 (e.g., the channel 424 and retained in place such that at least a portion of a head of the cotton swab extends outward from the opening 426 at the first end 422A of the body 402 to function as the nib 410.

The channel 424 can be configured with one or more retention features 430A, 430B, etc. configured to retain the cotton swab in position. As an example, the retention features 430A, 430B can be portions of an interior surface of the channel 424 that are inwardly tapered (e.g., radially inward with respect to the longitudinal axis A. As an example, when the cotton swab is double-headed, a first retention feature 430A positioned adjacent to the first end 422A of the body 402 can retain a first end of the cotton swab (e.g., by a friction fit), while a second retention feature 430B distanced from the first end 422A of the body 402 can retain a second end of the cotton swab (e.g., by a friction fit). It can be appreciated, however, that in circumstances where the cotton swab is single headed, retention forces provided by the first retention feature 430A can be sufficient to maintain the single head of the cotton swab in place to function as the nib 410.

The cap 412 can be further configured to affix to the body to cover the nib 410 and nib mounting portion 406 of the body 402. As discussed above, the cap 412 can protect the nib 410 during post production packaging, shipping, use and can prevent excessive evaporation of embodiments of the EO, enhancing shelf life of product. In certain embodiments, the cap can be secured to body by a clip lock mechanism or mating screw threads. Other mechanisms are also envisioned and can be employed without limit.

The configuration of the residue collection device 410 to allow the use of cotton swabs to function as the nib 410 provides a variety of benefits. As an example, the cotton swabs can be easily removed after use and replaced, allowing for the residue collection device 400 to be reused.

The residue collection device and the cotton swabs can be provided as a kit. As an example, the cotton swabs can be pre-wetted and individually enclosed within a hermetically sealed container. Under this configuration, a pre-wetted cotton swab can be inserted within the body 402 immediately prior to use for residue collection. In another example, cotton swabs can be enclosed individually or collectively in a dry state and the kit can further include a container holding the liquid solvent and an applicator (e.g., a dropper bottle filled with the liquid solvent, a bottle filled with the liquid solvent and a syringe applicator, a syringe applicator filled with the liquid solvent, etc.). Under this configuration, the liquid solvent can be dispensed by the applicator onto the cotton swab before or after insertion within the body 402 and immediately prior to use for residue collection.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

In the present disclosure, the words “a” or “an” are to be taken to include both the singular and the plural. Conversely, any reference to plural items shall, where appropriate, include the singular. In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

1. A sample residue collection device comprising:
a housing including a body, a nib housing, and an enclosure cap,
a nib, and
a liquid or solvent configured for receipt within the nib,
wherein the device is configured for detection and identification of samples residues by colorimetric indications.

2. The device in accordance with claim 1, further comprising a solvent reservoir positioned within the body and containing at least a portion of the liquid or solvent, wherein the solvent reservoir is in fluid communication with the nib.

3. The device in accordance with claim 1, wherein the device is in the form of a pen.

4. The device in accordance with claim 2, wherein the pen is composed of plastic and metal and combinations thereof.

5. The device in accordance with claim 2, wherein the body, is hollow and wherein the solvent reservoir in the form of a capillary or bibulous carrier is positioned within the body.

6. The device accordance with claim 2, wherein the solvent reservoir comprises a capillary composed of natural and/or synthetic fibers.

7. The device accordance with claim 6, wherein the capillary comprises at least one of wool, cotton, cellulosics, synthetic fibers, rayon, nylon or esters.

8. The device in accordance with claim 1, wherein the liquid or solvent comprises at least one of water, dimethyl sulphoxide, aliphatic and aromatic alcohols, essential oils, or terpenes.

9. The device in accordance with claim 8, wherein, the solvents comprise an essential oil.

10. The device in accordance with claim 9, wherein the essential oil comprises a Eucalyptus sp. Oil including at least one of Globulus, Blue Mallee, Lemon, or Radiata.

11. The device in accordance with claim 8, wherein the solvents comprise a melaleucal oil

12. The device in accordance with claim 11, wherein the melaleucal oil comprise a Melaleuca Alternifolia oil.

13. The device in accordance with claim 5, wherein the nib housing connects into the body, thereby supporting and holding the capillary in place.

14. The device in accordance with claim 1, wherein the nib is formed from a porous material comprising synthetic fibers, or natural fibers that allow saturation and wicking of solvents and encapsulation of sample residue.

15. The device in accordance with claim 1, wherein the nib is constructed at least in part of one or more of wool, cotton, cellulosics, synthetic fibers, rayon, nylon, esters, polyester, thermally bonded fibers, porous ceramics and/or metals.

16. The device in accordance with claim 1, wherein a tip of the nib is formed in a shape of a bullet, slanted chisel, flat chisel, cone, or step down bullet.

17. The device in accordance with claim 1, wherein the nib is inserted into and supported by the nib housing, protruding in such a way as to allow dabbing and rubbing enabling residue sample collection.

18. The device in accordance with claim 1, wherein the nib is removably coupled to the nib housing.

19. The device in accordance with claim 1, wherein the cap is configured to couple to the body, thereby covering the nib.

20. The device in accordance with claim 17, wherein the cap is configured to protect the nib during post manufacture packaging, shipping, storage and final use.

21. The device in accordance with claim 20, wherein the cap reduces evaporation of the solvent.

22. The device in accordance with claim 1, wherein the body and the nib housing and the body and the cap are fitted and configured to couple together by at least one of a male-female thread, pressure joining, or clip seal mechanisms.

23. The device in accordance with claim 1, wherein the safety of end users is greatly enhanced when sampling dangerous and hazardous substances as the need to scoop, wipe and perform multi-step sample collection methods by hand is reduced to a single step.

24. The device in accordance with claim 23, wherein the nib protrudes from the nib housing, extending inwards through the nib housing into the body and contacting the solvent reservoir which allows flow of solvent from the solvent reservoir into the nib and on contact of the wetted nib with a surface sample residue, wicking and dispersive forces allow the solvent to wet residue particulate preventing airborne particulate formation.

25. The device in accordance with claim 24, wherein end user safety is enhanced due to the prevention of airborne particulate landing on end user skin surfaces during sampling.

26. (canceled)
